(19) United States
(12) Patent Application Publication
(10) Pub. No.: US 2006/0002969 A1
(43) Pub. Date: Jan. 5, 2006

(54) Methods for reducing the foreign body reaction

(75) Inventors: Themis Kyriakides, Branford, CT (US); Paul Bornstein, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: University of Washington, Seattle, WA (US)

(21) Appl. No.: 11/044,640

(22) Filed: Jan. 26, 2005

Related U.S. Application Data
(60) Provisional application No. 60/539,821, filed on Jan. 27, 2004.

(51) Int. Cl. A61K 48/00; A61K 39/395; A61F 2/00

(52) U.S. Cl. 424/423; 424/146.1; 514/44

(57) ABSTRACT

The present invention provides methods for reducing the foreign body reaction against a structure implanted within an animal body. The methods include the step of providing an amount of an MMP-9 antagonist at the site of the implanted structure that is sufficient to reduce the foreign body response against the structure. The invention also provides implantable medical devices that include a surface layer that includes an amount of an MMP-9 antagonist sufficient to reduce the foreign body response against the device.

### CROSS-REFERENCE TO RELATED APPLICATION

The present application claims benefit of priority of U.S. Provisional Patent Application No. 60/539,821, filed Jan. 27, 2004.

### STATEMENT OF GOVERNMENT LICENSE RIGHTS

The U.S. Government has a paid-up license in this invention and the right in limited circumstances to require the patent owner to license others on reasonable terms as provided for by the terms of Grant No. EEC-9529161 awarded by the National Science Foundation.

### FIELD OF THE INVENTION

The present invention relates to methods for reducing the foreign body reaction against an implanted medical device.

### BACKGROUND OF THE INVENTION

Animals exhibit a variety of physiological and biochemical responses at the site of tissue damage or injury. These physiological and biochemical responses are collectively referred to as the wound response. The wound response facilitates the repair or replacement of the damaged or destroyed tissue. In some situations, however, wounded tissue exhibits a chronic wound response that adversely affects the health or well being of the wounded animal.

The implantation of a medical device into soft tissue elicits a wound response called the foreign body response. The foreign body response is initiated by deposition of proteins from plasma and tissue onto the surface of an implanted structure (e.g., an implanted medical device). Macrophages in the surrounding tissue investigate the abnormal, protein-coated, surface of the implanted structure and initiate a frustrated phagocytic response in which the macrophages attempt to engulf and digest the implanted structure. Typically, the macrophages fuse to form Foreign Body Giant Cells (abbreviated as FBGC), remain close to the surface of the implanted structure, and cause degradation of the implanted structure.

The foreign body response results in the encapsulation of the implant by a poorly-vascularized, collagenous, capsule that can compromise the function of the implant (e.g., compromise the function of an implanted medical device, such as a stent or drug delivery device). In addition, the continued presence of the implant can lead to a chronic inflammatory response. Thus, there is a continued need for methods and devices to reduce, or eliminate, the foreign body response against a medical device implanted into a living body.

### SUMMARY OF THE INVENTION

The present inventors have discovered that reduction of the amount, or inhibition of the biological activity, of matrix metalloproteinase-9 (abbreviated as MMP-9) causes a reduction in the foreign body response against a structure (e.g., medical device) implanted into the body of a living animal. The matrix metalloproteinases (MMPs) are zinc-dependent endopeptidases of the Metzincins superfamily. MMP-9 is one of two MMPs referred to as gelatinases. They have specific domain structures, minimally consisting of a propeptide, a catalytic domain, and a hemopexin-like domain. In addition to having the prototypic structure of MMPs, MMP-9 contains fibronectin type II-like repeats within their catalytic domain, resulting in a higher binding affinity to gelatin and elastin. MMP-9 also contains a type V collagen-like domain that is highly glycosylated, which may affect substrate specificity and resistance to degradation. MMP-9 is released from cells as a proenzyme. MMP-9 is activated, in vivo, via a protease cascade that results in cleavage of the prodomain. (MMP-9s are reviewed by Overall C. M., Methods Mol. Biol. 151:79-120 (2001) which publication is incorporated herein by reference).

In one aspect, the present invention provides methods for reducing the foreign body reaction against a structure implanted within an animal body. The methods of this aspect of the invention include the step of providing an amount of an MMP-9 antagonist at the site of the implanted structure that is sufficient to reduce the foreign body response against the structure. The methods of this aspect of the invention are applicable to any animal, including mammals, such as human beings. The methods of the invention can be used, for example, to reduce the foreign body reaction against a medical device (e.g., artificial hip joint, vascular stent, artificial lens) implanted within a mammalian body, thereby prolonging the working life of the medical device within the body.

In another aspect, the present invention provides implantable medical devices that each include (a) a device body; and (b) a surface layer attached to the device body, the surface layer comprising an amount of an MMP-9 antagonist sufficient to reduce the foreign body response against the device, wherein the device is adapted to be completely or partially implanted within an animal. The medical devices of this aspect of the invention can be implanted into animals, such as mammals (e.g., human beings). The reduction in the foreign body response against the implanted medical device helps to extend the working lifetime of the device within the body.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a perspective view of a representative medical device of the invention with a portion of the surface layer removed to expose the underlying device body.

FIG. 2 shows a transverse cross-section of the medical device of FIG. 1.

FIG. 3 shows the porous matrix structure of the surface layer of the representative medical device shown in FIG. 1.

### DETAILED DESCRIPTION OF THE PREFERRED EMBODIMENT

Unless specifically defined herein, all terms used herein have the same meaning as they would to one skilled in the art of the present invention. The following definitions are provided in order to provide clarity with respect to the terms as they are used in the specification and claims to describe the present invention.

The term “antibody” encompasses polyclonal and monoclonal antibodies, CDR-grafted antibodies, as well as hybrid antibodies, altered antibodies, F(AB)′₂ fragments, F(AB) molecules, Fv fragments, single domain antibodies, chimeric antibodies and functional fragments thereof which exhibit immunological binding properties of the parent antibody molecule. The antibodies can also be humanized.

The term “foreign body response” refers to a type of wound response in which a poorly-vascularized, collagenous, capsule forms around a structure (such as a medical device) implanted into an animal body.

The term “hybridize under stringent conditions”, and grammatical equivalents thereof, refers to the ability of a nucleic acid molecule to hybridize to a target nucleic acid molecule (such as a target nucleic acid molecule immobilized on a DNA or RNA blot, such as a Southern blot or Northern blot) under defined conditions of temperature and salt concentration. Typically, stringent hybridization conditions are no more than 25° C. to 30° C. (for example, 10° C.) below the melting temperature (Tm) of the native duplex. By way of non-limiting example, representative salt and temperature conditions for achieving stringent hybridization are: 5×SSC, at 65° C., or equivalent conditions; see generally, Sambrook et al. Molecular Cloning. A Laboratory Manual, 2nd ed., Cold Spring Harbor Press, 1987; Ausubel et al., Current Protocols in Molecular Biology, Greene Publishing, 1987. Tm for nucleic acid molecules greater than about 100 bases can be calculated by the formula Tm=0.5+0.41%(G+C)−log (Na⁺). For oligonucleotide molecules less than 100 bases in length, exemplary hybridization conditions are 5 to 10° C. below Tm. On average, the Tm of a short oligonucleotide duplex is reduced by approximately (500/oligonucleotide length)° C.

As used herein, the term “implantable medical device” refers to a medical device that is adapted to be implanted into the body of an animal, such as a mammal, and which operates within the body of the animal. The device may be completely or partially implanted into the body of an animal.

The term “sequence identity” or “percent identical” as applied to nucleic acid molecules is the percentage of nucleic acid residues in a candidate nucleic acid molecule sequence that are identical with a subject nucleic acid molecule sequence, after aligning the sequences to achieve the maximum percent identity, and not considering any nucleic acid residue substitutions as part of the sequence identity. No gaps are introduced into the candidate nucleic acid sequence in order to achieve the best alignment.

Nucleic acid sequence identity can be determined in the following manner. The subject polynucleotide molecule sequence is used to search a nucleic acid sequence database, such as the GenBank database (maintained by the National Center for Biotechnology Information, U.S. National Library of Medicine, 8600 Rockville Pike, Bethesda, Md. 20894), using the program BLASTN version 2.1 (based on Altschul et al., Nucleic Acids Research 25:3389-3402 (1997)). The program is used in the ungapped mode. Default filtering is used to remove sequence homologies due to regions of low complexity as defined in Wootton, J. C., and S. Federhen, Methods in Enzymology 266:554-571 (1996). The default parameters of BLASTN are utilized.

The term “sequence identity” or “percent identical” as applied to protein molecules is the percentage of amino acid residues in a candidate protein molecule sequence that are identical with a subject protein sequence, after aligning the sequences to achieve the maximum percent identity. No gaps are introduced into the candidate protein sequence in order to achieve the best alignment.

Amino acid sequence identity can be determined in the following manner. The subject protein sequence is used to search a protein sequence database, such as the GenBank database, using the BLASTP program. The program is used in the ungapped mode. Default filtering is used to remove sequence homologies due to regions of low complexity. The default parameters of BLASTP are utilized. Filtering for sequences of low complexity utilize the SEG program.

The abbreviation “SSC” refers to a buffer used in nucleic acid hybridization solutions. One liter of the 20× (twenty times concentrate) stock SSC buffer solution (pH 7.0) contains 175.3 g sodium chloride and 88.2 g sodium citrate.

In one aspect, the present invention provides methods for reducing the foreign body reaction against a structure implanted within an animal body. The methods of this aspect of the invention include the step of providing an amount of an MMP-9 antagonist at the site of the implanted structure that is sufficient to reduce the foreign body response against the structure. The methods of this aspect of the invention are applicable to any animal, including mammals, such as human beings. The present invention can be used, for example, to reduce the foreign body reaction against an implantable medical device that has been implanted into an animal body.

The phrase “reducing the foreign body response”, and grammatical equivalents thereof, includes reducing the magnitude of one or more of the biochemical and/or physiological and/or physical responses that make up the foreign body response, and/or reducing the duration of the foreign body response. A reduction in the foreign body response is characterized by at least one of the following changes in a component of the foreign body response that occurs as a result of treatment of animal tissue in accordance with the methods of the invention: a decrease in the amount of fibrosis (measured, for example, by a decrease in hydroxy-proline content which indicates the level of collagen in the foreign body capsule); a decrease in the amount of inflammation (measured, for example, by counting the number of inflammatory cells, and the number of foreign body giant cells, in histological sections; or measuring the levels of cytokines, such as interleukin and monocyte chemo-attractant protein, in wound extracts by ELISA); an increase in the amount of vascularization of the capsule formed as part of the foreign body response (measured, for example, by visualizing blood vessels in histological sections with anti-PECAM1 antibody and the peroxidase reaction; the number of vessels and their average size are estimated with imaging software such as Metamorph (sold by Universal Imaging Corporation, 402 Boot Road, Downingtown, Pa. 19335, U.S.A.)); an increase in the amount of permeability of the capsule formed as part of the foreign body response (measured, for example, as the release of traceable chemicals from implanted devices, or ability of implanted sensors to sense plasma levels of molecules such as glucose); a decrease in the amount of the capsule formed around the foreign body (capsule thickness can be measured from histological sections with the aid of ocular micrometers); and a decrease in the amount of contraction of collagen fibers within the capsule that is formed as part of the foreign body response (measured as tensile strength of the capsule or induced shape change on malleable implants). The decrease, or increase, of any of the foregoing parameters can be a decrease, or increase, relative to the amount of the parameter present before treatment in accordance with the methods of the invention; or a decrease, or increase, relative to the amount of the parameter present in control tissue that is not treated in accordance with the methods of the present invention.

Representative MMP-9 antagonists include: non-nucleic acid molecules that inhibit the activity of metalloproteinases, such as MMP-9; MMP-9 antisense nucleic acid molecules (such as antisense oligonucleotides); siRNA molecules targeted against an MMP-9 gene or mRNA; anti-MMP-9 antibodies; and blocking peptides which interact with MMP-9 or an MMP-9 receptor, thereby preventing MMP-9 from eliciting a biological response.

An MMP-9 antisense nucleic acid molecule may be constructed in a number of different ways provided that it is capable of interfering with the expression of a target gene. For example, an antisense nucleic acid molecule can be constructed by inverting the coding region (or a portion thereof) of MMP-9 relative to its normal orientation for transcription to allow the transcription of its complement.

The antisense nucleic acid molecule is usually substantially identical to at least a portion of the target gene or genes. The antisense nucleic acid molecule, however, need not be perfectly identical to inhibit expression. Generally, higher homology can be used to compensate for the use of a shorter antisense nucleic acid molecule. The minimal percent identity is typically greater than about 65%, but a higher percent identity may exert a more effective repression of expression of the endogenous sequence. Substantially greater percent identity of more than about 80% typically is preferred, though about 95% to absolute identity is typically most preferred.

The antisense nucleic acid molecule need not have the same intron or exon pattern as the target gene, and non-coding segments of the target gene may be equally effective in achieving antisense suppression of target gene expression as coding segments. A DNA sequence of at least about 30 or 40 nucleotides may be used as the antisense nucleic acid molecule, although a longer sequence is preferable. In the present invention, a representative example of a useful antagonist of MMP-9 is an antisense MMP-9 nucleic acid molecule which is at least ninety percent identical to the complement of the human MMP-9 cDNA consisting of the nucleic acid sequence set forth in SEQ ID NO:1. The nucleic acid sequence set forth in SEQ ID NO:1 encodes the MMP-9 protein consisting of the amino acid sequence set forth in SEQ ID NO:2.

Other representative examples of useful antagonists of MMP-9 are antisense MMP-9 nucleic acid molecules that are at least 2000 nucleotides long, and that hybridize to the nucleic acid molecule having the sequence set forth in SEQ ID NO:1 under conditions of 5×SSC at 50° C. for 12 hours followed by washing in 5×SSC at 60° C. for 1 hour. Sodium dodecyl sulfate may be included in the hybridization and wash solutions at a concentration of 0. 1% (w/v).

Other representative examples of useful antagonists of MMP-9 are antisense MMP-9 nucleic acid molecules that are at least 500 nucleotides long, and that hybridize to the nucleic acid molecule having the sequence set forth in SEQ ID NO:1 under conditions of 5×SSC at 50° C. for 12 hours followed by washing in 5×SSC at 60° C. for 1 hour. Sodium dodecyl sulfate may be included in the hybridization and wash solutions at a concentration of 0.1% (w/v).

MMP-9 antisense oligonucleotides may be used to reduce the level of MMP-9 protein synthesis. Typically an antisense oligonucleotide is at least 15 nucleotides long (more typically from 20 to 100 nucleotides long) and is complementary to a portion of an MMP-9 gene or mRNA. Thus, examples of antisense oligonucleotides that are useful in the practice of the present invention are from 15 to 100 nucleotides long and are complementary to a portion (of equal length to the antisense oligonucleotide) of the human MMP-9 cDNA consisting of the nucleic acid sequence set forth in SEQ ID NO:1.

The following are representative examples of antisense nucleic acid molecules useful for antisense inhibition of the synthesis of MMP-9: (1) 5′ GCT CAT TGG TGA GGG CAG AGG 3′ (SEQ ID NO:3) (disclosed by London, C. A., et al., Cancer Gene Therapy 10:823-832 (2003) (this is a cyclical morpholino oligonucleotide); 5′CUCAUGGUGAGGGCAGAGGUGUCU-3′ (SEQ ID NO:4) (disclosed by Spessotto, P., et al., J Cell Biol. 158:1133-44 (2002)); and 5′ CTG CCA GGG ACT CAT GCG AAA GC 3′ (SEQ ID NO:5) (disclosed by Whiteside, E. J., et al., Biol. Reprod. 64:1331-1337 (2001)). Additionally, a complete antisense MMP-9 cDNA was used to inhibit MMP-9 synthesis in DU-145 carcinoma cells (Manes, S., J. Biol. Chem. 274(11):6935-6945 (1999)); and a 528-bp antisense MMP-9 cDNA fragment was used to inhibit synthesis of MMP-9 in human glioblastoma cells (Kondraganti, S., et al., Cancer Research 60:6851-6855 (2000)). Each of the foregoing publications describing antisense inhibition of MMP-9 synthesis is incorporated herein by reference.

In another embodiment of this aspect of the present invention, the MMP-9 antagonist is an anti-MMP-9 antibody. By way of representative example, antigen useful for raising antibodies can be prepared in the following manner. A nucleic acid molecule (such as an MMP-9 cDNA molecule) is cloned into a plasmid vector, such as a Bluescript plasmid (available from Stratagene, Inc., La Jolla, Calif.). The recombinant vector is then introduced into an E. coli strain (such as E. coli XL1-Blue, also available from Stratagene, Inc.) and the polypeptide encoded by the nucleic acid molecule is expressed in E. coli and then purified.

Polypeptides can also be prepared using peptide synthesis methods that are well known in the art. The synthetic polypeptides can then be used to prepare antibodies. Direct peptide synthesis using solid-phase techniques (Stewart et al., Solid-Phase Peptide Synthesis, W H Freeman Co, San Francisco Calif. (1969); Merrifield, J. Am. Chem. Soc. 85:2149-2154 (1963) can be used instead of recombinant or chimeric peptide production. Automated synthesis may be achieved, for example, using Applied Biosystems 431 A Peptide Synthesizer in accordance with the instructions provided by the manufacturer. Methods for preparing monoclonal and polyclonal antibodies are well known to those of ordinary skill in the art and are set forth, for example, in chapters five and six of Antibodies A Laboratory Manual, E. Harlow and D. Lane, Cold Spring Harbor Laboratory (1988). Antibody production includes not only the stimulation of an immune response by injection into animals, but also analogous processes such as the production of synthetic antibodies, the screening of recombinant immunoglobulin libraries for specific-binding molecules (Orlandi et al., Proc. Nat'l. Acad. Sci. USA 86:3833, 1989, or Huse et al. Science 256:1275, 1989), or the in vitro stimulation of lymphocyte populations.

An example of a commercially available anti-MMP-9 antibody is the anti-human MMP-9 monoclonal antibody (raised in mouse) sold by Chemicon International (catalog number MAB13415). Another example of a commercially available anti-MMP-9 antibody is the anti-mouse MMP-9 monoclonal antibody (raised in rat) from R&D Systems, Inc. (614 McKinley Place N.E., Minneapolis, Minn. 55413; catalog number MAB9091).

siRNA molecules are also useful for reducing the level of expression of MMP-9. siRNAs are double stranded RNA molecules having a length of 21-22 nucleotides. An siRNA selectively silences genes that include a sequence that is complementary to the sequence of one of the strands of the siRNA molecule. siRNAs are reviewed, for example, by C. D. Novina and P. A. Sharp, Nature 430:161-164 (2004), which publication is incorporated by reference herein in its entirety. Examples of siRNA molecules that are useful in the practice of the present invention to reduce the level of expression of MMP-9 include siRNA molecules that include a 21 or 22 nucleotide sequence that is complementary to a 21 or 22 nucleotide sequence present in the human MMP-9 cDNA consisting of the nucleic acid sequence set forth in SEQ ID NO:1. Specific examples of commercially available anti-MMP-9 siRNAs are available from Ambion Inc., 2130 Woodward, Austin, Tex. 78744-1832, U.S.A.

The following anti-MMP-9 siRNAs (identified by their Ambion product number) are directed against a rat MMP-9 gene (GenBank accession number NM 031055): Ambion product numbers 53119, 53210, and 53299. The following anti-MMP-9 siRNAs (identified by their Ambion product number) are directed against a human MMP-9 gene (GenBank accession number NM 004994): Ambion product numbers 42846, 45007, and 45096. The following anti-MMP-9 siRNAs (identified by their Ambion product number) are directed against a mouse MMP-9 gene (GenBank accession number NM 013599): Ambion product numbers 71047, 71142, and 71233.

Successful siRNA inhibition of MMP-9 has been reported by Sance'au, J., et al. (J. Biological Chemistry 278(38):36537-36546 (2003)), which publication is incorporated herein by reference. The siRNA molecules targeted nucleotides 377-403 (numbered relative to the first nucleotide of the start codon of the MMP-9 open reading frame) of human MMP-9 (GenBank accession number NM-004994). The nucleotide sequence of the target sequence was 5′-AAC ATC ACC TAT TGG ATC CAA ACT AC-3′) (SEQ ID NO:6). The sense si-RNA sequence was 5′-CAUCACCUAUUGGAUCCAAdTdT-3′ (SEQ ID NO:7). The antisense si-RNA was 5′-UUGGAUCCAAUAGGUGAUGdTdT-3′ (SEQ ID NO:8) (wherein “dT” stands for deoxythymidine).

siRNA inhibition of human MMP-9, in vivo, has also been reported by Lakka, S. S., et al., Oncogene 23:4681-4689 (2004), which publication is incorporated herein by reference. Lakka et al. used an siRNA molecule having a sense strand sequence of 5′UCAAGUGGCACCACCACAACAUdTdT 3′ (SEQ ID NO:9), and an antisense strand sequence of 5′ dTdTAGUUCACCGUGGUGGUGUUGUA 3′ (SEQ ID NO:10). The MMP-9 siRNA targeted the region at nucleotides 360 to 381 of the MMP-9 mRNA (5′ TCAAGTGGCACCACCACAACAT 3′ (SEQ ID NO:11)).

Non-nucleic acid molecules that inhibit the action of metalloproteinases are also useful for reducing the level of expression of MMP-9. The following are examples of useful non-nucleic acid molecules that inhibit the action of metalloproteinases, such as MMP-9: Z-PDLDA-NHOH (Z-Pro-D-Leu-D-Ala-NHOH) (sold by Calbiochem, 10394 Pacific Center Court, San Diego, Calif. 92121, U.S.A., catalog number 234140); MMP Inhibitor II (N-Hydroxy-1,3-di-(4-methoxybenzenesulphonyl)-5,5-dimethyl-[1,3]-piperazine-2-carboxamide) (sold by Calbiochem, catalog number 444247); MMP Inhibitor IV (HONH—COCH₂CH₂CO—FA-NH₂) (sold by Calbiochem, catalog number 444271); MMP-2/MMP-9 Inhibitor I ((2R)-2-[(4-Biphenylylsulfonyl)amino]-3-phenylpropionic Acid) (sold by Calbiochem, catalog number 444241); MMP-2/MMP-9 Inhibitor II ((2R)-[(4-Biphenylylsulfonyl)amino]-N-hydroxy-3-phenylpropionamide) (sold by Calbiochem, catalog number 444249); MMP-2/MMP-9 Inhibitor IV (SB-3CT) (sold by Calbiochem, catalog number 444274); MMP-9/MMP-13 Inhibitor I (N-Hydroxy-1-(4-methoxyphenyl)sulfonyl-4-(4-biphenylcarbonyl)piperazine-2-carboxamide) (sold by Calbiochem, catalog number 444252); MMP-9/MMP- 13 Inhibitor II (N-Hydroxy-1-(4-methoxyphenyl)sulfonyl-4-benzyloxycarbonylpiperazine-2-carboxamide) (sold by Calbiochem, catalog number 444253); GM6001 MMP Inhibitor (sold by CHEMICON International, Inc., 28820 Single Oak Drive, Temecula, Calif. 92590, U.S.A., catalog number CC1000); Batimastat (BB94) (sold by Vernalis plc, Oakdene Court, 613 Reading Road, Winnersh, RG41 5UA, U.K.); Marimastat (BB 2516) (sold by Vernalis plc); Neovastat (AE-941) (sold by AEterna Zentaris Inc., 1405 boul. du Parc-Technologique, Québec, Québec G1P 4P5, Canada); and Prinomastat AG3340 (sold by Pfizer Inc, 235 East 42nd Street, New York, N.Y. 10017, U.S.A.)

Another useful antagonist of MMP-9 is TIMP-1, which is a member of the Tissue Inhibitors of Matrix Metalloproteinases (TIMPs) family of proteins. The TIMPs all have affinity for MMP-9, but typically MMP-9 is secreted by cells as part of a noncovalent complex with TIMP-1. TIMP-1 complexes with the carboxy-terminal of the proenzyme form of MMP-9 as well as the catalytic domain of the active form of MMP-9. The antagonistic effect of TIMP-1 on MMP-9 is described in Lambert et al., Crit. Rev. Oncol. Hematol. 49:187-198 (2004), which publication is incorporated herein by reference. Exemplary nucleic acid molecules that encode TIMP-1 proteins hybridize to the complement of the TIMP-1 cDNA set forth in SEQ ID NO:12 under stringent hybridization conditions. For example, some nucleic acid molecules that encode a TIMP-1 protein hybridize to the complement of the TIMP-1 cDNA set forth in SEQ ID NO:12 under conditions of 5×SSC at 50° C. for 12 hours followed by washing in 5×SSC at 60° C. for 1 hour. Sodium dodecyl sulfate may be included in the hybridization and wash solutions at a concentration of 0.1% (w/v). Again by way of example, some nucleic acid molecules that encode a TIMP-1 protein hybridize to the complement of the TIMP-1 cDNA set forth in SEQ ID NO:12 under conditions of 5×SSC at 50° C. for 12 hours followed by washing in 5×SSC at 55° C. for 1 hour. Sodium dodecyl sulfate may be included in the hybridization and wash solutions at a concentration of 0.1% (w/v).

Exemplary TIMP-1 proteins that are useful in the practice of the present invention include TIMP-1 proteins that are at least 70% identical (e.g., at least 80% identical, or at least 90% identical, or at least 95% identical, or at least 99% identical) to the exemplary TIMP-1 protein having the amino acid sequence set forth in SEQ ID NO:13.

MMP-9 antagonist can be provided at the site of a structure implanted into a living animal body by any useful method. For example, MMP-9 antagonist can be injected into the area of the animal body surrounding an implanted structure, such as a medical device. Again by way of example, MMP-9 antagonist molecules can be immobilized onto a surface of a structure, such as an implantable medical device. The modified surface will typically be in contact with living tissue after implantation into an animal body. Such implantable medical devices can be made from, for example, nitrocellulose, diazocellulose, glass, polystyrene, polyvinylchloride, polypropylene, polyethylene, dextran, Sepharose, agar, starch, nylon or metal. Linkage of the MMP-9 antagonist to a surface can be accomplished by any technique that does not destroy the biological activity of the linked MMP-9 antagonist. A surface of a structure, such as a surface of an implantable medical device, can be modified to include functional groups (e.g., carboxyl, amide, amino, ether, hydroxyl, cyano, nitrido, sulfanamido, acetylinic, epoxide, silanic, anhydric, succinimic, azido) for immobilization of MMP-9 antagonist thereto. Coupling chemistries include, but are not limited to, the formation of esters, ethers, amides, azido and sulfanamido derivatives, cyanate, and other linkages to the functional groups available on MMP-9 antagonists. Additionally, MMP-9 antagonists can be incorporated into a gel or porous matrix on the surface of an implantable medical device.

Examples of effective concentrations and dosages for selected antagonists of MMP-9 include: for antibodies a dose of about 1 mg/kg, of the body weight of the animal, administered once every 3 days to 4 days is useful, or an equivalent cumulative dose of about 1 mg/kg, of the body weight of the animal, administered continuously over a three day to four day period; for antisense and siRNA an effective concentration is about 25 nanomoles which is constantly maintained at the site of implantation during the life of the implanted structure (e.g., an implanted medical device can include a surface layer that continually releases antisense and/or siRNA molecules in an amount sufficient to achieve a concentration of about 25 nanomoles in the tissue surrounding the implanted medical device). An example of an effective daily dose of TIMP-1 is at least 10 micrograms/day.

The following examples of effective concentrations of selected non-nucleic acid molecules, that inhibit the action of metalloproteinases, are set forth in brackets after the name of the inhibitor: Z-PDLDA-NHOH (1 micromolar); MMP Inhibitor II (3 nanomolar); MMP Inhibitor IV (HONH—COCH₂CH₂CO—FA-NH₂) (500 nanomolar); MMP-2/MMP-9 Inhibitor I ((2R)-2-[(4-Biphenylylsulfonyl)amino]-3-phenylpropionic Acid) (250 nanomolar); MMP-2/MMP-9 Inhibitor II ((2R)-[(4-Biphenylylsulfonyl)amino]-N-hydroxy-3-phenylpropionamide) (30 nanomolar); MMP-2/MMP-9 Inhibitor IV (SB-3CT) (600 nanomolar); MMP-9/MMP-13 Inhibitor I (N-Hydroxy-1-(4-methoxyphenyl)sulfonyl-4-(4-biphenylcarbonyl)piperazine-2-carboxamide) (900 picomolar); MMP-9/MMP-13 Inhibitor II (N-Hydroxy-1-(4-methoxyphenyl)sulfonyl-4-benzyloxycarbonylpiperazine-2-carboxamide) (2 nanomolar); GM6001 MMP Inhibitor (0.1 nanomolar); Batimastat (BB94) (0.5 micromolar); Marimastat (BB 2516) (1.0 micromolar); Neovastat (AE-941) (40 nanomolar); and Prinomastat AG3340 (5.0 micromolar). Thus, for example, in some embodiments of the methods of the present invention, a constant effective concentration of MMP-9 antagonist (e.g., 5.0 micromolar Prinomastat) is maintained at the site of an implanted device within an animal body.

In another aspect, the present invention provides implantable medical devices that each include (a) a device body; and (b) a surface layer attached to the device body, the surface layer comprising an amount of an MMP-9 antagonist sufficient to reduce a foreign body response against the device when the device is implanted into a living animal body, wherein the device is adapted to be completely or partially implanted within an animal. The medical devices of this aspect of the invention can be implanted into animals, such as mammals (e.g., human beings). The medical devices of this aspect of the invention can be used, for example, in the practice of the methods of the present invention wherein the surface layer of the device provides an amount of MMP-9 antagonist sufficient to reduce the foreign body response against the implanted medical device.

Implantable medical devices of the invention may be completely implanted into an animal body (i.e., the entire device is implanted within the body), or the device may be partially implanted into an animal body (i.e., only part of the device is implanted within an animal body, the remainder of the device being located outside of the animal body). Representative examples of completely implantable medical devices include, but are not limited to: cardiovascular devices (such as vascular grafts and stents), artificial blood vessels, artificial lenses, artificial bone joints, such as hip joints, and scaffolds that support tissue growth (in such anatomical structures as nerves, pancreas, eye and muscle). Representative examples of partially implantable medical devices include: biosensors (such as those used to monitor the level of drugs within a living body, or the level of blood glucose in a diabetic patient) and percutaneous devices (such as catheters) that penetrate the skin and link a living body to a medical device, such as a kidney dialysis machine.

The device body can be made from any suitable material (or mixtures or combinations thereof), including natural materials, synthetic materials, plastics, metals and alloys. Representative examples of synthetic polymers useful for making the device body include: (poly)urethane, (poly)carbonate, (poly)ethylene, (poly)propylene, (poly)lactic acid, (poly)galactic acid, (poly)acrylamide, (poly)methyl methacrylate, and (poly)styrene. Useful natural polymers include collagen, hyaluronic acid, and elastin.

The surface layer can cover the whole of the device body, or one or more parts of the device body, such as areas of the device body where it is desired to reduce the foreign body response. The surface layer can be made, for example, from any suitable material that (a) permits deposition therein, or attachment thereto, of an amount of an MMP-9 antagonist sufficient to reduce the foreign body response against the medical device; and (b) can be attached to the device body (before or after deposition within, or attachment to, the surface layer of an amount of an MMP-9 antagonist sufficient to reduce the foreign body response against the medical device). Representative examples of materials useful for making the surface layer include porous matrices. Porous matrices are useful, for example, for delivering antisense MMP-9 molecules to an animal body.

Representative porous matrices useful for making the surface layer are those prepared from tendon or dermal collagen, as may be obtained from a variety of commercial sources, (e.g., Sigma and Collagen Corporation), or collagen matrices prepared as described in U.S. Pat. Nos. 4,394,370 and 4,975,527. One collagenous material is termed UltraFiber™, and is obtainable from Norian Corp. (Mountain View, Calif., U.S.A.)

Certain polymeric matrices may also be employed if desired, these include acrylic ester polymers and lactic acid polymers, as disclosed, for example, in U.S. Pat. Nos. 4,526,909 and 4,563,489. Particular examples of useful polymers are those of orthoesters, anhydrides, propylene-cofumarates, or a polymer of one or more α-hydroxy carboxylic acid monomers, (e.g., α-hydroxy acetic acid (glycolic acid) and/or α-hydroxy propionic acid (lactic acid).

The surface layer can be made, for example, by attachment of MMP-9 antagonist to the device body, for example by covalent activation of the surface of the medical device. By way of representative example, MMP-9 antagonist can be attached to the device body by any of the following pairs of reactive groups (one member of the pair being present on the surface of the device body, and the other member of the pair being present on the MMP-9 antagonist: hydroxyl/carboxylic acid to yield an ester linkage; hydroxyl/anhydride to yield an ester linkage; hydroxyl/isocyanate to yield a urethane linkage.

A surface of a device body that does not possess useful reactive groups can, for example, be treated with radio-frequency discharge plasma (RFGD) etching to generate reactive groups in order to allow deposition of MMP-9 antagonist (e.g., treatment with oxygen plasma to introduce oxygen-containing groups; treatment with propyl amino plasma to introduce amine groups). When an RFGD glow discharge plasma is created using an organic vapor, deposition of a polymeric overlayer occurs on the exposed surface. RFGD plasma deposited films offer several unique advantages. They are smooth, conformal, and uniform. Film thickness is easily controlled and ultra-thin films (10-1000 Angstroms thick) are readily achieved, allowing for surface modification of a material without alteration to its bulk properties. Moreover, plasma films are highly-crosslinked and pin-hole free, and therefore chemically stable and mechanically durable. RFGD plasma deposition of organic thin films has been used in microelectronic fabrication, adhesion promotion, corrosion protection, permeation control, as well as biomaterials. (see, e.g., Ratner, U.S. Pat. No. 6,131,580).

An amount of an MMP-9 antagonist sufficient to reduce the foreign body response to the implanted medical device is included in or on a surface layer of the medical device. Examples of MMP-9 antagonists are described herein in connection with the methods of the present invention for reducing the foreign body reaction against a structure implanted within an animal body. Any combination of MMP-9 antagonists can be included in, or on, a surface layer of a medical device of the invention. Examples of effective concentrations of the representative MMP-9 antagonists are also described herein in connection with the methods of the present invention for reducing the foreign body reaction against a structure implanted within an animal body. Thus, during the functional lifetime of an implanted medical device of the present invention, the implanted device may continuously provide an effective amount of an MMP-9 antagonist to the region of the animal body surrounding the implanted device (e.g., continuously provide Prinomastat at a concentration of 5.0 micromolar in the region in contact with the implanted medical device).

MMP-9 antagonists can be covalently linked to a surface of the device body, or can be non-covalently linked (e.g., releasably disposed within a gel or porous matrix on a surface of the device body). For example, anti-MMP-9 antibodies and/or TIMP-1 molecules can be covalently linked to a surface of the device body. Again by way of example, antisense oligonucleotides, and/or siRNA molecules, and/or non-nucleotide antagonists of MMP-9 can be releasably disposed within a gel or porous matrix on a surface of the device body (the MMP-9 antagonists can diffuse out of the gel or porous matrix, into the surrounding tissue, over time). Again by way of example, nucleic acid plasmids, or other expression vectors, that encode an MMP-9 antisense molecule or anti-MMP-9 siRNA molecule, can be releasably disposed within a gel or porous matrix on a surface of the device body. The expression vector molecules are released from the gel or porous matrix over time and are taken up by cells surrounding the implanted device and expressed within the cells, thereby inhibiting expression of MMP-9 within the cells.

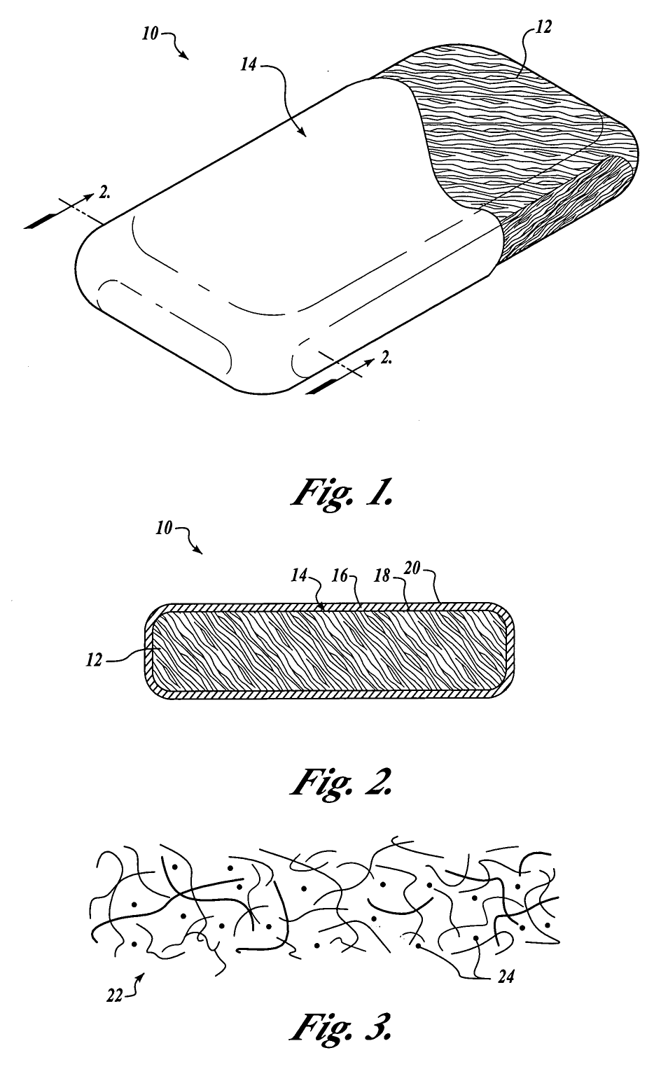

FIG. 1 shows a representative medical device of the present invention, in the form of an implantable drug delivery device 10, which includes a device body 12 to which is attached a surface layer 14. In the embodiment shown in FIG. 1, surface layer 14 has been partially removed to show device body 12 beneath. Device body 12 is indicated by hatching. As shown in the cross-sectional view of medical device 10 in FIG. 2, surface layer 14 includes a surface layer body 16 that defines an internal surface 18, attached to device body 12, and an external surface 20.

In the representative embodiment of device 10 shown in FIGS. 1 and 2, surface layer 14 is made from a porous matrix 22. FIG. 3 shows a representation of porous matrix 22 within which are disposed molecules 24 of an antagonist of MMP-9 (other molecules, such as drugs, may also be disposed within porous matrix 22). Thus, in operation, device 10 is implanted into an animal body where molecules 24 are released into the surrounding tissue over time, and reduce the foreign body response by the animal body against implanted device 10. Drug delivery device 10 is sized appropriately; for example the length of drug delivery device 10 may be about 1 cm, the width of drug delivery device 10 may be about 0.5 cm, and the height of drug delivery device 10 may be about 0.25 cm.

The following examples merely illustrate the best mode now contemplated for practicing the invention, but should not be construed to limit the invention.

### EXAMPLE 1

This Example shows that the foreign body response is reduced in genetically altered mice that do not produce MMP-9 (MMP-9 null mice). In particular, the number of foreign body giant cells (FBGC) produced by MMP-9 null mice in response to implanted cellulose ester filters was significantly less than the number of FBGC produced by control mice that did produce MMP-9. Also, the collagen fibers that were deposited around the implanted filters were more loosely organized in the MMP-9 mice than in the control mice.

Rat anti-mouse Mac 3 antibody was obtained from BD Pharmingen (San Diego, Calif.). A Vectastain ABC kit for immunohistochemistry was purchased from Vector Laboratories (Burlingame, Calif.). Filters (0.45-μm pore size, mixed cellulose ester) were purchased from Millipore (290 Concord Rd., Billerica, Mass. 01821, U.S.A.) 25 mm² Millipore filters were soaked in 95% ethanol for 24 hours, rinsed extensively with phosphate-buffered saline (PBS), and stored in endotoxin-free phosphate buffered saline (PBS) until implantation.

Subcutaneous implantation of the mixed cellulose ester filters was performed in accordance with the regulations adopted by the National Institutes of Health and approved by the Animal Care and Use Committee of the University of Washington. A total of 16 MMP-9-null mice and 24 control mice were used for implantations. Each mouse received two implants, providing for eight implants per group per time point. The genetic background of MMP-9-null mice was 93% C57BL/6 and 7% SJL, and of control mice, C57BL/6.

Explanated biomaterials were excised after four weeks and fixed in 10% zinc-buffered formalin for 24 hours. Following processing and embedding, 6-μm sections (eight per sample) were stained with hematoxylin and eosin (H&E) and Masson's trichrome. Capsule thickness was measured at 10 different locations (five per side) with the aid of an ocular micrometer. Immunolocation of Mac3 (1:500) was performed according to the supplier's instructions. All secondary peroxidase-conjugated Ab were used at 1:200 dilution. Controls included sections treated with pre-immune sera. Sections from MMP-9-null mice served as a negative control for the immunodetection of MMP-9. The number of FBGC was estimated from H&E- and Mac3-stained sections (eight per sample). The number of nuclei per FBGC was estimated from the analysis of over 200 cells for control and over 100 cells for MMP-9-null mice.

The MMP-9-null mice mounted a normal inflammatory response as judged by the presence of numerous cells involved in the inflammatory response. Immunohistochemical analysis of the filters with the anti-Mac 3 antibody (which specifically binds to macrophages and foreign body giant cells (FBGC)) revealed a significant reduction in the formation of FBGC in MMP-9 knockout mice (0.1±0.007 FBGC per visual field) when compared to control mice (3.9±3.1 FBGC per visual field).

The thickness of encapsulation of the filters in MMP-9 knockout mice (96±32 mm) was similar to the thickness of encapsulation of the filters in control mice (82±30 mm). The capsules formed in the MMP-9 knockout mice, however, consisted of loosely organized collagen fibers and appeared to be significantly less dense than the capsules formed in the control mice.

### EXAMPLE 2

This Example shows that binding MMP-9 with anti-MMP-9 antibodies, in vitro, inhibits formation of FBGC from monocytes (monocytes differentiate to produce macrophages which, in turn, fuse to form FBGC).

Freshly isolated human peripheral blood monocytes were plated (1×10⁶ cells per well) and induced to fuse by the addition of IL-4 (10 ng/ml) and GM-CSF (10 ng/ml) on day 3 and 7 of culture. On day 10, wells were stained (May-Grunwald) to visualize cells. In addition to IL-4 and MG-CSF, wells received either 5 μg of anti-MMP9 antibody or 5 μg isotype control antibody (mouse IgG). The anti-MMP-9 antibody was an anti-human MMP-9 monoclonal antibody (raised in mouse) obtained from Chemicon International (catalog number MAB13415).

Wells treated with anti-MMP9 antibody exhibited reduced FBGC formation (3.7±1.3 FBGC/high power field) in comparison to isotype-treated wells (17.9±6.2/high power field). In addition, the number of nuclei per foreign body giant cell was less in the anti-MMP9-treated cells (6.4±2.6) compared to isotype treated cells (11.9±2.1). In isotype IgG-treated wells almost all the cells fused (85±1.8%), whereas in the anti-MMP9-treated wells only a small fraction of the cells fused (9.8±4.9%) (p value≦0.05; n=12 for all comparisons).

### EXAMPLE 3

This Example shows that anti-MMP-9 antibodies inhibit FBGC formation in vivo.

Cellulose ester filters (0.5 μm pore size, sold by Millipore) were implanted into the peritoneal cavity of wild-type mice (3 month old, 25 gram weight). Under normal conditions numerous FBGC form on the surface of an implant within 10-14 days of implantation. To block MMP-9 function, 25 μg anti-MMP-9 antibody or isotype control antibody (rat IgG), in 500 μl saline, were injected intraperitoneally at 2 days and 8 days following implantation. Implants were harvested on day 14 after implantation and processed for histological examination. The anti-MMP-9 antibody was an anti-mouse MMP-9 monoclonal antibody (raised in rat) from R&D Systems, Inc. (614 McKinley Place N.E., Minneapolis, Minn. 55413; catalog number MAB9091).

Examination of hematoxylin and eosin-stained histological sections indicated that treatment of animals with the anti-MMP-9 antibody resulted in a significant reduction of FBGC formation in comparison to isotype antibody-treated animals (0.63±0.49 versus 2.93±0.86 FBGC/200 μm; p value≦0.05; n=5).

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. An implantable medical device comprising:
(a) a device body; and
(b) a surface layer attached to the device body, the surface layer comprising an amount of an MMP-9 antagonist sufficient to reduce the foreign body response against the device, wherein the device is adapted to be completely or partially implanted within an animal.

2. An implantable medical device of claim 1 wherein the implantable medical device is selected from the group consisting of a vascular graft, a stent, an artificial blood vessel, an artificial lens, an artificial bone joint, a biosensor, a catheter, and artificial skin.

3. An implantable medical device of claim 1 wherein the layer completely covers the body of the device.

4. An implantable medical device of claim 1 wherein the layer partially covers the body of the device.

5. An implantable medical device of claim 1 wherein the MMP-9 antagonist is selected from the group consisting of antisense MMP-9 nucleic acid molecules, anti-MMP-9 antibodies, anti-MMP-9 siRNA molecules and non-nucleic acid antagonists of MMP-9.

6. An implantable medical device of claim 1 wherein the surface layer comprises a porous matrix and a member of the group consisting of antisense MMP-9 nucleic acid molecules, anti-MMP-9 antibodies, anti-MMP-9 siRNA molecules and non-nucleic acid antagonists of MMP-9 is releasably disposed within the porous matrix.

7. An implantable medical device of claim 1 wherein a member of the group consisting of antisense MMP-9 nucleic acid molecules, anti-MMP-9 antibodies, anti-MMP-9 siRNA molecules and non-nucleic acid antagonists of MMP-9 is covalently attached to the device body.

8. A method of reducing the foreign body reaction against a structure implanted within an animal body, the method comprising the step of providing an amount of an MMP-9 antagonist, at the site of the implanted structure, that is sufficient to reduce the foreign body response against the structure.

9. A method of claim 8 wherein the structure is an implantable medical device adapted to be affixed to, or implanted within, the soft tissue of an animal.

10. A method of claim 9 wherein the implantable medical device is selected from the group consisting of a vascular graft, a stent, an artificial blood vessel, an artificial lens, an artificial bone joint, a biosensor, a catheter, and artificial skin.

11. A method of claim 8 wherein the MMP-9 antagonist is selected from the group consisting of antisense MMP-9 nucleic acid molecules, anti-MMP-9 antibodies, anti-MMP-9 siRNA molecules and non-nucleic acid antagonists of MMP-9.

12. A method of claim 8 wherein the MMP-9 antagonist is non-covalently attached to an external surface of the structure.

13. A method of claim 8 wherein the MMP-9 antagonist is covalently attached to an external surface of the structure.

14. A method of claim 8 wherein the MMP-9 antagonist is releasably disposed within a porous matrix attached to an external surface of the structure.

15. A method of claim 8 wherein the MMP-9 antagonist is releasably disposed within a gel attached to an external surface of the structure.

16. A method of claim 8 wherein the MMP-9 antagonist is injected into a region of the body surrounding the implanted structure.

17. A method of claim 8 wherein the body is a human body.


SEQUENCE LISTING (13 sequences)

SEQ ID NO: 1 - DNA, 2334 bp, Homo Sapiens
  CDS (20)..(2143)
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 2 - protein, 707 aa, Homo Sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 3 - DNA, 21 bp, Artificial Sequence
  Antisense Inhibition of MMP-9 Synthesis
gctcattggtgagggcagagg

SEQ ID NO: 4 - RNA, 24 nt, Artificial Sequence
  Antisense Inhibition of MMP-9 Synthesis
cucauggugagggcagaggugucu

SEQ ID NO: 5 - DNA, 23 bp, Artificial Sequence
  Antisense Inhibition of MMP-9 Synthesis
ctgccagggactcatgcgaaagc

SEQ ID NO: 6 - DNA, 26 bp, Homo Sapiens
aacatcacctattggatccaaactac

SEQ ID NO: 7 - RNA, 21 nt, Artificial Sequence
  Oligonucleotide
caucaccuauuggauccaann

SEQ ID NO: 8 - RNA, 21 nt, Artificial Sequence
  Oligonucleotide
uuggauccaauaggugaugnn

SEQ ID NO: 9 - RNA, 24 nt, Artificial Sequence
  Oligonucleotide
ucaaguggcaccaccacaacaunn

SEQ ID NO: 10 - RNA, 24 nt, Artificial Sequence
  Oligonucleotide
nnaguucaccguggugguguugua

SEQ ID NO: 11 - DNA, 22 bp, Homo Sapiens
tcaagtggcaccaccacaacat

SEQ ID NO: 12 - DNA, 782 bp, Homo Sapiens
  CDS (63)..(386)
aggggccttagcgtgccgcatcgccgagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagtttcgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaactgaagcctgcacagtgtccaccctgttcccactcccatctttcttccggacaatgaaataaagagttaccacccagc

SEQ ID NO: 13 - protein, 108 aa, Homo Sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLI